(19) United States
(12) Patent Application Publication
(10) Pub. No.: US 2026/0152782 A1
(43) Pub. Date: Jun. 4, 2026

(54) RAPID NUCLEIC ACID EXTRACTION FROM BIOFLUIDS

(71) Applicants: Siril Arockiam, Tempe, AZ (US); Karen Anderson, Scottsdale, AZ (US)

(72) Inventors: Siril Arockiam, Tempe, AZ (US); Karen Anderson, Scottsdale, AZ (US)

(21) Appl. No.: 19/408,332

(22) Filed: Dec. 3, 2025

Related U.S. Application Data
(60) Provisional application No. 63/727,595, filed on Dec. 3, 2024.

(51) Int. Cl. C12Q 1/6806; C12N 15/10; C12Q 1/6844; C12Q 1/70

(52) U.S. Cl. CPC C12Q 1/6806; C12N 15/1006; C12Q 1/6844; C12Q 1/701

(57) ABSTRACT

Disclosed herein are novel buffers for rapid and simplified nucleic acid extraction, and methods of their use. The buffers comprise a chaotropic agent, such as guanidine hydrochloride, sodium chloride, or lithium chloride, combined with Tris-EDTA buffer at a specific pH. The buffers facilitate efficient lysis of cells and viruses, inactivate nucleases, and enable binding of nucleic acids to various silica surfaces without the need for multiple wash steps or specialized equipment.

### CROSS-REFERENCE TO RELATED APPLICATIONS

This application claims the benefit of and priority to U.S. Provisional Application No. 63/727,595 filed on Dec. 3, 2024, the content of which is incorporated by reference in its entirety

### STATEMENT REGARDING FEDERALLY SPONSORED RESEARCH OR DEVELOPMENT

This invention was made with government support under CA211415 awarded by the National Institutes of Health. The government has certain rights in the invention.

### BACKGROUND

Nucleic acid extraction from biofluids such as saliva, nasal swabs, urine, blood, and cervical fluid is the first step for molecular diagnostics. Sample preparation involving purification and concentration of nucleic acid is crucial for applications such as pathogen detection, gene expression analysis, cancer screening, sequencing, and genetic research. The COVID-19 pandemic has showcased the importance of nucleic acid amplification testing (NAAT) for diagnosing and preventing of transmission of infections. The recent pandemic also highlighted the limitations of centralized testing laboratories for sample transport and long turnaround time for rapid detection of infectious diseases. As a result, inexpensive and rapid point-of-care (POC) diagnostics for NAAT of infections such as SARS-CoV-2, respiratory syncytial virus (RSV), and influenza are needed to manage outbreaks by testing at the site of healthcare providers and mobile clinics.

Traditional nucleic acid (NA) extraction broadly falls into chemical, solid-phase, and mechanical methods. Extraction methods, such as phenol-chloroform, paramagnetic beads, and silica matrices, are sensitive but time-consuming, labor-intensive, and require sophisticated laboratory infrastructure and sample storage. These factors significantly impact the deployment of molecular diagnostics at the point of care, especially in low-resource settings, emphasizing the need for a simpler, rapid, and instrument-free extraction method. To address this, fully integrated sample-to-answer portable instruments such as GeneXpert®, and Cobas® have been widely used but remain cost limiting for widespread adaptation, specifically in low- and middle-income countries.

Many respiratory pathogens are detected in saliva. RNA and DNA viruses such as SARS-CoV-2, RSV, influenza, zika virus, HPV, herpes virus, human immunodeficiency virus, and Epstein-Barr virus can be diagnosed using saliva. Saliva, as a non-invasive sample collection biofluid, makes it ideal for self-collection to detect pathogens and use for sequencing. However, saliva presents unique challenges for DNA/RNA extraction in POC. It has variable viscosity, potential inhibitory substances, food particles, and inconsistent sample quality, necessitating a tailored approach for sample preparation. Direct NA extraction methods using heat, proteinase K or detergents like Triton X-100 or Tween-20 can result in less sensitive and variable nucleic acid yields due to RNA degradation through high RNase activity in saliva compared to other biofluids. Methods using magnetic or silica beads often need a pre-centrifugation step to remove sputum or heavy particulates. Hence, a simple, robust, and integrable sample preparation method is needed to extract, stabilize, concentrate, and purify NA from different biofluids, specifically in saliva for POC systems.

Lab-on-a-chip extraction systems, with innovations like paper-based extraction, centrifugal devices using hand-powered mechanisms or fidget spinners, membrane filters, xurography, and dielectrophoresis, provide portable, compact, and disposable sample preparation systems in POC. These microfluidic devices integrate complex laboratory processes into a compact, user-friendly format. Isothermal amplifications such as loop-mediated amplification (LAMP), recombinase polymerase amplification (RPA), and helicase-dependent amplification (HDA) are emerging as alternatives to PCR in POC diagnostics. Among these, the combination of simple, efficient nucleic acid extraction methods with LAMP represents a significant advancement. However, the translation of these technologies in fully integrated POC NAAT diagnostics faces challenges, such as complex fabrication design, metering systems, motorized pumps, multiple wash chambers with active actuators, and sample dispensers. Additionally, there is a need for sample pretreatment, and customized NA extraction methods for each type of biofluid. There remains a significant gap in the market for a method that combines speed, simplicity, efficiency, and compatibility with a range of downstream molecular detection techniques.

### SUMMARY

In a first aspect, provided herein is a composition for extracting nucleic acids from a sample, the composition comprising: a chaotropic agent at a concentration of between 1M and about 4M; and a Tris-EDTA buffer comprising about 10 mM Tris-HCl and between about 0.1 and about 10 mM EDTA.

In embodiments, the chaotropic agent is guanidine hydrochloride (GuHCl); and the GuHCl may be at a concentration of between about 1 M and about 2 M. In embodiments, the chaotropic agent is sodium chloride (NaCl) or lithium chloride (LiCl). The EDTA may be at a concentration of about 1 mM.

The composition may further comprise tris(2-carboxyethyl)phosphine TCEP at a concentration of between about 1 mM and about 100 mM. The TCEP may be at a concentration of about 10 mM.

A pH of the composition may be between about 6 and about 8. In embodiments, the pH is about 7.

The composition may further comprise at least one of an RNase inhibitor and a proteinase.

In another aspect, provided herein is a kit comprising: the composition described herein; a carrier RNA; and a precipitating agent.

The precipitating agent may be ethanol or polyethylene-glycol (PEG).

In another aspect, provided herein is a method for extracting nucleic acids from a biological sample, the method comprising: a) combining equal volumes of the sample with the composition described herein to create a mixture; b) incubating the mixture at between about 55° C. and about 95° C. for between about 30 seconds to about 10 minutes; c) cooling the mixture to room temperature; and d) separating the nucleic acids from the mixture.

The method may further comprise adding a carrier RNA to the mixture before step b). The method may further comprise adding PEG at a concentration of about 10% w/v to the mixture before step b). The method may further comprise adding ethanol or isopropanol to the mixture at a ratio of about 1:1 after step c) and before step d); wherein the ethanol is about 100% ethanol; and wherein the isopropanol is about 25% to about 100% isopropanol. Step b) may be performed at about 95° C. for about 5 minutes.

Step d) may comprise adding the mixture to an extraction agent to separate the nucleic acids from the remaining mixture; discarding the remaining mixture; adding an elution buffer to the extraction agent to create an eluate comprising the nucleic acids; and collecting the eluate.

The extraction agent may be selected from paramagnetic silica beads, glass fiber sheets, and glass silica beads. The method may further comprise analyzing the nucleic acids using at least one of loop-mediated amplification (LAMP), recombinase polymerase amplification (RPA), quantitative PCR, and nucleic acid sequencing.

The biological sample may comprise saliva.

### BRIEF DESCRIPTION OF THE DRAWINGS

FIGS. 1A-1C are schematic diagrams of three types of nucleic acid extraction/purification techniques. (A) shows a classic method of nucleic acid isolation using a spin-column based DNA/RNA extraction with multiple wash steps. (B) shows a simplified point of care purification (POC-pure) rapid DNA/RNA extraction method with no wash steps to adopt for on-chip nucleic acid extraction. (C) shows point of care (POC) nucleic acid extraction workflow using the centrifuge-free on-chip extraction of a nucleic acid microfluidic chip. The nucleic acid can be detected using loop-mediated isothermal amplification (LAMP), with either colorimetry or fluorescence detection. Colorimetric detection requires only a portable heater and microfluidic nucleic acid extraction chip.

FIGS. 2A-2F show the development of custom buffers for total nucleic acid (NA) binding to silica. All the LAMP reactions were performed in triplicate for SARS-CoV-2 target and amplified for 60 minutes. The mean time to detection was plotted on all graphs with the y-axis error bars extending one standard deviation. (A) shows the titration of Guanidine Hydrochloride (GuHCl), from 0-10 M in 1×TE (Tris HCl-EDTA) buffer to find the optimum concentration for RNA binding and LAMP PCR inhibition due to carryover buffer. For all extractions, 50,000 (50K) synthetic SARS CoV-2 (Omicron) template was spiked in 200 μL of water. (B) shows the titration of GuHCl, from 0-10 M in 1×TE (Tris HCl-EDTA) with carrier RNA (5.6 μg) added to enhance binding. (C) shows the titration of GuHCl, from 0-10 M in 1×TE (Tris HCl-EDTA) with ethanol added in a 1:1 volume ratio to buffer to enhance binding. (D) shows the titration of GuHCl, from 0-10 M in 1×TE (Tris HCl-EDTA) with 5.6 μg carrier RNA and 1:1 volume ethanol. (E) shows the titration of 2M GuHCL buffer with 0-200% (v/v) of 100% ethanol. (F) shows the optimum binding for NA with 2M GuHCL buffer and 1:1 (100%) ethanol titrated at pH 6-10.

FIGS. 3A-3B show optimization of viral lysis and nucleic acid elution conditions. (A) shows a pooled negative saliva sample (200 μL) spiked with 50K heat inactivated SARS CoV-2 (Omicron) virus particles. All samples were treated with 200 μL of lysis agents and heated at 95° C. for 5 minutes. All the samples were extracted using the custom buffer and extraction protocol. LAMP PCR was performed with orf1a as target for 60 minutes. (B) shows different elution buffer conditions evaluated for LAMP inhibition, RNA retrieval, and degradation. All LAMP reactions were performed in triplicate for the SARS CoV-2 target. The mean time to detection was plotted on all graphs with the y-axis error bars extending one standard deviation.

FIGS. 4A-4B show results of RNase inactivation for saliva samples. (A) shows an RNaseAlert™ assay (ThermoFisher) performed for different salivary treatment methods to find the optimum nuclease inactivation method. Saliva (200 μL) was treated with 200 μL of different buffers and heat-inactivated at 95° C. for 5 minutes. Samples were then cooled, and the relative fluorescence unit (RFU) was measured after 30 minutes of incubation at 37° C. using the RNaseAlert™ assay to find the RNase activity. All assays were performed in triplicate and mean RFU was plotted with the y-axis error bars extending one standard deviation. (B) shows a scatter line plot for RNase activity observed from 0-30 mins at 2-min intervals. The subset on the right shows the magnified version of RNase activity for conditions with low RFU. Of all the methods, custom buffer (CB)+10 mM TCEP showed the lowest RNase activity and was chosen as an optimum RNase inactivation condition for salivary samples.

FIGS. 5A-5F show a comparison of nucleic acid extraction efficiency between PureLink™ and POC-Pure methods. All extractions and no template control (NTC) were performed in triplicate. The mean time to detection was plotted on all graphs with the y-axis error bars extending one standard deviation. The data points are annotated with numbers in parentheses to show amplified replicates. If not shown, all the replicates were amplified for that particular copy number. ND=no detected fluorescence signal at the end of 60 minutes of amplification. (A) shows for RNA, 50,000 (50K) heat inactivated SARS CoV-2 (Omicron) virus particles spiked in 200 μL saliva sample and extracted using PureLink™ (ThermoFisher) and POC-Pure. Taqman qPCR for SARS CoV-2 was performed for both extracts to compare extraction efficiency. (B) shows extracts from FIG. 5A also amplified with LAMP PCR for SARS-CoV-2 (RNA) and HPV16 (DNA) for 60 mins and the time to detection plotted. (C) shows results of the Qubit™ extraction performed to compare the DNA yield using PureLink™ and POC-Pure methods. (D) shows the limit of detection (LOD) for SARS-CoV-2 spiked in 200 μL 1×TE buffer using the POC-Pure method. (E) shows LOD for heat inactivated SARS CoV-2 (Omicron) virus spiked in 200 μL saliva. (F) shows LOD for HPV16 DNA spiked in 200 μL specimen transport medium (STM).

FIGS. 6A-6D show on-chip extraction using customized rapid extraction with a microfluidic chip. (A) shows a schematic of a microfluidic chip fabricated in the lab for centrifuge-free total nucleic acid (NA) extraction. The silica embedded in the microfluidic channel is used to capture and purify NA. (B) shows an image of a fully fabricated and functional microfluidic chip with a sample syringe attached to the sample port and a valve actuator. The clip can be pushed towards or away from the valve actuator to redirect the fluid flow between the waste reservoir and elution port. (C) shows on-chip extraction of 50,000 SARS CoV-2 virus spiked in 200 μL saliva and SARS-CoV-2 RNA detection using LAMP for five saliva samples. Three saliva samples were spiked with a SARS-CoV-2 template. Negative salivary samples did not show amplification for the SARS-CoV-2 target. (D) shows on-chip extraction of 5,000 SiHa cells spiked in 200 μL specimen transport medium (STM) and HPV16 DNA detection using LAMP for 3 different samples and no template control (NTC). NTC did not amplify for either HPV16E6 or ACTB targets, suggesting no carryover or cross-contamination. ND=No fluorescence signal detected at the end of 60 minutes of amplification.

FIGS. 7A-7B show comparisons of volume and time reduction using various nucleic acid extraction methods. (A) shows comparisons of the waste volume generated for a 200 μL sample input for PureLink™, DNeasy Kit, POC-Pure, and on-chip methods. The POC-Pure method results in reduction of more than 50% of the waste volume, while retaining similar efficacy. (B) shows comparisons of overall extraction time for one sample at a time. On-chip extraction takes less than 10 minutes, an approximate 60% reduction in time compared to commercial methods, due to less heating time and reduced number of steps.

FIG. 8 shows titration of GuHCl from 0-10 M in 1×TE buffer with 5.6 μg carrier RNA and 1:1 volume isopropanol.

### DETAILED DESCRIPTION

The present disclosure is a rapid and simplified method for extracting nucleic acids (DNA and RNA) from biological samples, customized for point-of-care (POC) molecular diagnostics and use in resource-limited settings. This innovative approach employs a custom buffer system that effectively lyses cells and viruses, inactivates nucleases, and enables nucleic acid binding to various silica-based materials-such as paramagnetic silica beads, glass fiber sheets, and silica glass beads-without the need for multiple wash steps or specialized equipment. By eliminating complex procedures and reducing extraction time to approximately 10 minutes, the method streamlines the nucleic acid extraction process while maintaining high yield and integrity of the genetic material. The extracted nucleic acids are compatible with a range of downstream molecular detection techniques, including isothermal amplification (e.g., loop-mediated isothermal amplification or LAMP), quantitative PCR (qPCR), and sequencing. The method's adaptability allows for the substitution of reagents based on availability, such as using alternative salts or polyethylene glycol (PEG) in place of ethanol, enhancing its applicability in diverse settings. This disclosure holds significant commercial potential by enabling the development of rapid, cost-effective, and reliable diagnostic tools that can be deployed at the point of care. Its implementation can greatly benefit healthcare providers by facilitating timely disease detection and management, particularly in remote or resource-constrained environments.

In a first aspect, provided herein is a method for extracting nucleic acids from a sample, the composition comprising: a chaotropic agent at a concentration of between 1M and about 4M; and a tris (hydroxymethyl)aminomethane-ethylenediaminetetraacetic acid (Tris-EDTA) buffer comprising about 10 mM tris-hydrochloric acid (Tris-HCl) and between about 0.1 and about 10 mM EDTA.

A chaotropic agent is a molecule in water solution that can disrupt the hydrogen bonding network between water molecules. This affects the stability of the native state of other molecules in the solution, mainly macromolecules, such as proteins and nucleic acids, by weakening the hydrophobic effect. In exemplary embodiments, the chaotropic agent is guanidine hydrochloride (GuHCl). The GuHCl may be at a concentration of between about 1 M and about 2 M. In other embodiments the chaotropic agent is sodium chloride (NaCl) or lithium chloride (LiCl).

As used herein, the terms “sample” and “biological sample” refer to a sample taken from a subject. Suitable samples include fluid samples (e.g., saliva, blood, serum, plasma, urine, stool, cerebrospinal fluid) and tissue samples. In exemplary embodiments, the sample comprises saliva.

The EDTA may be at a concentration of about 1 mM.

The composition may further comprise tris(2-carboxyethyl)phosphine TCEP at a concentration of between about 1 mM and about 100 mM. The TCEP may be at a concentration of about 10 mM.

The composition may be at a pH of between 6 and 8. In exemplary embodiments, the ph of the composition is about 7.

The composition may further comprise at least one of a ribonuclease (RNase) inhibitor and a proteinase. RNase inhibitors are proteins that bind to and inactivate ribonucleases to protect RNA from degradation. Proteinases are enzymes that break down proteins by hydrolyzing peptide bonds. In exemplary embodiments, the proteinase is proteinase K.

In a second aspect, provided herein is a kit comprising any one of the compositions described herein; a carrier RNA; and a precipitating agent. A carrier RNA is an inert nucleic acid added to solutions during nucleic acid purification to improve the recovery of low-concentration DNA or RNA. A precipitating agent is a reagent that is added to a solution to cause a solid/precipitate to form. In exemplary embodiments, the precipitating agent is ethanol. In other embodiments, the precipitating agent is isopropanol or polyethylene glycol (PEG).

The kit may further comprise an elution buffer. The elution buffer may comprise nuclease-free water or AE buffer. AE buffer typically comprises a 5 mM Tris-HCl solution at a pH of between about 8.5 and about 9, with a specific concentration of 10 mM Tris-Cl and 0.5 mM EDTA.

In a third aspect, provided herein is a method of extracting nucleic acids from a biological sample, the method comprising: a) combining equal volumes of the sample with any one of the compositions described herein to create a mixture; b) incubating the mixture at between about 55° C. and about 95° C. for between about 30 seconds to about 10 minutes; c) cooling the mixture to room temperature; and d) separating the nucleic acids from the mixture.

The method may further comprise adding a carrier RNA to the mixture before step b).

In embodiments, the method further comprises adding PEG at a concentration of about 10% w/v to the mixture before step b). In embodiments, the method further comprising adding ethanol or isopropanol to the mixture at a ratio of about 1:1 after step c) and before step d). The ethanol may be about 100% ethanol. The isopropanol may be about 25% to about 100% isopropanol.

Step b) may be performed at about 95° C. for about 5 minutes.

Step d) may comprise adding the mixture to an extraction agent to separate the nucleic acids from the remaining mixture; discarding the remaining mixture; adding an elution buffer to the extraction agent to create an eluate comprising the nucleic acids; and collecting the eluate. An extraction agent is a solid support suitable for binding nucleic acids. Suitable extraction agents include, but are not limited to, paramagnetic silica beads, glass fiber sheets, and glass silica beads.

The method may further comprise analyzing the nucleic acids using at least one of loop-mediated amplification (LAMP), recombinase polymerase amplification (RPA), quantitative PCR, and nucleic acid sequencing.

The sample may comprise saliva.

### Components of the Custom Buffer (POC-Pure)

Chaotropic Agent: Guanidine Hydrochloride (GuHCl): Effective concentration range from 1 M to 4 M, with 1-2 M being optimal. Alternative Salts: Sodium chloride (NaCl) or lithium chloride (LiCl) can be used as substitutes at the same concentration range (0 M to 4 M).
Buffer System: Tris-EDTA (TE) Buffer: Comprising 10 mM Tris-HCl and 0.1-10 mM EDTA, adjusted to a pH range of 6 to 8, preferably at pH 7.0.
Reducing Agent (Optional): Tris(2-carboxyethyl)phosphine (TCEP): Used at a concentration range of 1 mM to 100 mM, with 10 mM being preferred, to inactivate nucleases, particularly RNases.
Carrier RNA (Optional): Added to enhance nucleic acid binding and protect target nucleic acids from degradation. Typical amounts range from 1 μg to 10 μg per 200 μL of sample.

### Precipitating Agent:

Ethanol: Added post-heating at a volume ratio ranging from 0% to 200% of the sample volume, preferably a 1:1 ratio (equal volume).
Polyethylene Glycol (PEG): Used as an alternative to ethanol, added before the heating step at a final concentration of 5% to 15% (w/v), with 10% (w/v) being optimal.
Suitable Silica Surfaces for Nucleic Acid Binding: Paramagnetic Silica Beads: Allow for magnetic separation without centrifugation. Glass Fiber Sheets: Provide a matrix for nucleic acid binding and can be incorporated into filtration devices. Silica Glass Beads: Offer a high surface area for nucleic acid adsorption. Other Commercially Available or Synthesized Silica Matrices.

### Step-by-Step Procedure for Nucleic Acid Extraction:

1. Sample Collection and Preparation: Collect the biological sample (e.g., saliva, blood, nasal swab, cervical fluid, skin lesion, oral rinse, sputum) in a sterile container. For saliva, collect approximately 200 μL of sample. The extraction method can handle 100 μL-2000 μL. Scale the following procedure based on following information.
2. Preparation of the Custom Buffer (POC-Pure):

For GuHCl-based Buffer: Dissolve 19.1 g of GuHCl in 70 mL of nuclease-free water. Add 1 mL of 1 M Tris-HCl (pH 7.0) and 200 μL of 0.5 M EDTA to reach final concentrations of 10 mM Tris-HCl and 1 mM EDTA. Adjust the volume to 100 mL with nuclease-free water. Adjust pH to 7.0 if necessary.

For Sodium or Lithium Chloride-based Buffer: Replace GuHCl with 11.7 g of NaCl or 17 g of LiCl to achieve 2 M concentration.

Optionally, add TCEP to the buffer to reach a final concentration of 10 mM. The buffer can be stored at room temperature until use.
3. Sample Lysis and Nuclease Inactivation:

Mixing: In a sterile tube, combine 200 μL of the biological sample with 200 μL of the custom buffer. If using carrier RNA, add 5.6 μg to the mixture. If using PEG as the precipitating agent, add it to achieve a final concentration of 10% (w/v) (e.g., add 40 μL of 50% PEG 8000 solution).

Homogenization: Vortex or mix the sample thoroughly for 30 seconds to ensure complete mixing.

Heating: Incubate the mixture at 95° C. for 5 minutes to lyse cells/viruses and inactivate nucleases. The inactivation time can range between 2 mins to 10 mins too. If using heat-sensitive components, adjust the temperature to a range between 55° C. to 95° C. as needed.

Cooling: Allow the mixture to cool to room temperature (approximately 5 minutes).
4. Nucleic Acid Binding:

If Using Ethanol: Add 200 μL of 100% ethanol to the cooled mixture (1:1 volume ratio). Mix thoroughly by inversion or gentle vortexing. Isopropanol can also be used at 25% to 100% volume

Binding to Silica Surface or Paramagnetic Silica Beads: Add an appropriate amount (e.g., 10 μL of bead suspension) to the mixture. Incubate at room temperature for 5 minutes with gentle mixing. Place the tube on a magnetic separator for 1-2 minutes until the beads collect on the side. Carefully remove and discard the supernatant without disturbing the beads.

Glass Fiber Sheets or Silica Glass Beads: Load the mixture onto a column or device containing the silica material. Allow the mixture to pass through by gravity, gentle vacuum, or centrifugation at 8,000×g for 1 minute. Collect the flow-through in a waste container.
5. Optional Wash Step (If Desired): If the extraction is performed on microfluidic chip, where there will be residual or dead volume on the channel or fluidic path. Elution buffer can be used to wash or displace the sample-buffer mixture.
6. Nucleic Acid Elution:

Elution Buffer: Use nuclease-free water for RNA or TE buffer (10 mM Tris-HCl, 0.5 mM EDTA, pH 9.0) for DNA. RNasecure™ reagent or DTT can also be added to elution to enhance the NA stability.

Elution Volume: Add 50-100 μL of elution buffer directly to the silica material or beads. It can be increased up to 1:1 sample input volume.

### Elution Process:

Paramagnetic Beads: Resuspend the beads in the elution buffer. Incubate at 37 C for 1-5 minutes or at room temperature for 10 minutes. Place on a magnetic separator and transfer the supernatant containing nucleic acids to a new tube.

Columns/Sheets/Beads: Apply the elution buffer to the silica surface. Incubate for 1-5 minutes.

Collect the eluate by centrifugation, gravity flow, or gentle vacuum into a clean tube.
7. Nucleic Acid Quantification and Quality Assessment (Optional):

Measure nucleic acid concentration using a spectrophotometer (e.g., NanoDrop™) or fluorometer (e.g., Qubit™). Assess purity by evaluating the A260/A280 and A260/A230 ratios.
8. Downstream Molecular Detection: The extracted nucleic acids are suitable for:

Isothermal Amplification (e.g., LAMP): Use 2-10 μL of the eluate in a standard LAMP reaction.

Quantitative PCR (qPCR): Use 2-5 μL of the eluate per reaction.

Sequencing: Follow standard library preparation protocols using the eluate.
9. POC: Integration into Microfluidic Devices for sample preparation by placing the silica membrane or matrix on the fluidic path and using the above optimized protocol.

The term “subject” may be used interchangeably with the terms “individual” and “patient” and includes human and non-human subjects.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In the context of the present disclosure, the following abbreviations for the commonly occurring nucleic acid bases are used. “A” refers to adenine, “C” refers to cytosine, “G” refers to guanine, “T” refers to thymine, and “U” refers to uracil. The aforementioned abbreviations may also be used to refer to nucleosides or nucleotides comprising the nucleic acid bases. For example, “G” may refer guanine, guanosine, or guanidine, depending on the context.

The terms “protein” or “polypeptide” or “peptide” are used interchangeably to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The cited references are incorporated by reference herein in their entireties. In the event that there is an inconsistency between a definition of a term in the specification as compared to a definition of the term in a cited reference, the term should be interpreted based on the definition in the specification.

### EXAMPLES

### Example 1. Rapid On-Chip Nucleic Acid Extraction for Point-of-Care Salivary Diagnostics

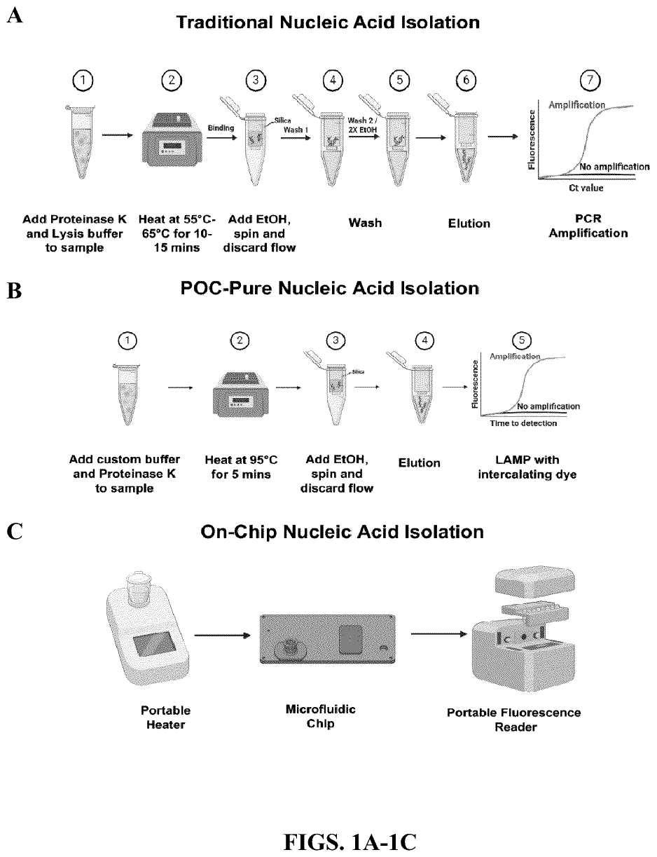

The present disclosure is related to a lab-on-a-chip adaptable custom extraction method called POC-Pure that addresses the limitations of current extraction techniques in POC settings. This is done by reducing the number of user steps, extraction time, and waste volume generated while also maintaining DNA/RNA stability, yield, and concentrating nucleic acids (NA) during the extraction process. This method is compatible with qPCR and LAMP, yielding similar target amplification times. Furthermore, purification leads to fewer carryover inhibitors from saliva for downstream applications. These factors are crucial for integrating nucleic acid extraction with isothermal amplification into a single sample-to-answer platform. We use silica membranes and a chaotropic salt, guanidine hydrochloride (GuHCl), to rapidly and efficiently bind NA to the silica 43.44. Additionally, we developed methods for POC RNase inactivation and RNA stabilization methods from saliva to ensure the yield of extracted RNA for sensitive salivary diagnostics. The schematic comparison (FIGS. 1A and 1B) highlights the streamlined and simplified steps involved in our POC-Pure extraction method, which eliminates multiple wash steps while retaining the efficiency of commercial extraction methods and maintaining the nucleic acid yield and amplification efficiency for SARS-CoV-2 RNA and HPV 16 DNA from biofluids.

### Results and Discussion

FIG. 1A illustrates the schematic workflows of classic nucleic acid (NA) purification and (FIG. 1B) shows the POC-Pure extraction method which eliminates multiple wash steps. FIG. 1C demonstrates the centrifuge-free on-chip NA extraction workflow for rapid point-of-care testing. The microfluidic device can purify and concentrate NA from salivary samples on-chip for rapid molecular diagnostic applications.

To develop a simplified and easy-to-adapt sample preparation and nucleic acid extraction method for POC applications, compatible with qPCR and isothermal amplification (LAMP), we first developed a custom buffer for silica membrane-based extraction and optimized salivary sample treatment. To reduce sample processing time and wash steps while retaining similar nucleic acid extraction efficiency and enabling on-chip translation, we addressed the following conditions for optimal DNA and RNA binding: (I) development of a custom silica binding buffer for total nucleic acid (NA), (II) optimization of viral lysis and NA elution conditions, (III) RNase inactivation in salivary samples, (IV) comparison of the custom method with a commercial extraction method, and (V) demonstration of the custom rapid extraction method on the microfluidic chip.

### 2.1 Custom Buffer for Nucleic Acid (NA) Binding

Guanidine hydrochloride (GuHCl) and guanidine thiocyanate (GuSCN) are often used at high concentrations in commercial DNA/RNA extraction kits due to their strong denaturant and chaotropic properties.^{45,46} Guanidinium has high ion affinity, making it an excellent candidate to form a salt bridge between silicon dioxide (silica) and nucleic acid. The positively charged salts help in the interaction between the negatively charged silica and the negatively charged phosphate backbones of NA. This selective yet reversible binding nature of silica and guanidinium helps remove unbound contaminants and proteins and aids in concentrating nucleic acids. Guanidinium is favored over others such as sodium, cesium, and potassium due to its rapid binding, strong chaotropic effect, ion affinity, and disruption of electrostatic interactions. Between GuHCl and GuSCN, we chose GuHCl in this study because trace amounts of GuHCl (<140 mM) can enhance the amplification efficiency of LAMP, making it an ideal candidate for a wash-free column-based extraction method.^{47,48}

### 2.1.1 Guanidine HCl and Carrier RNA

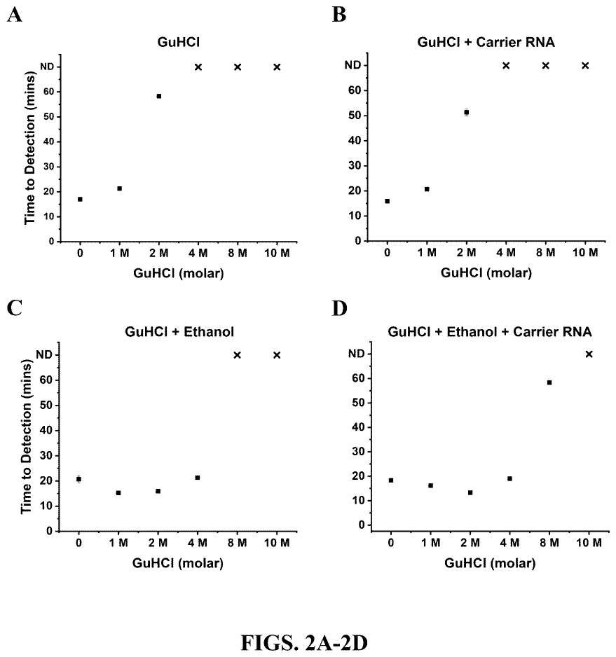

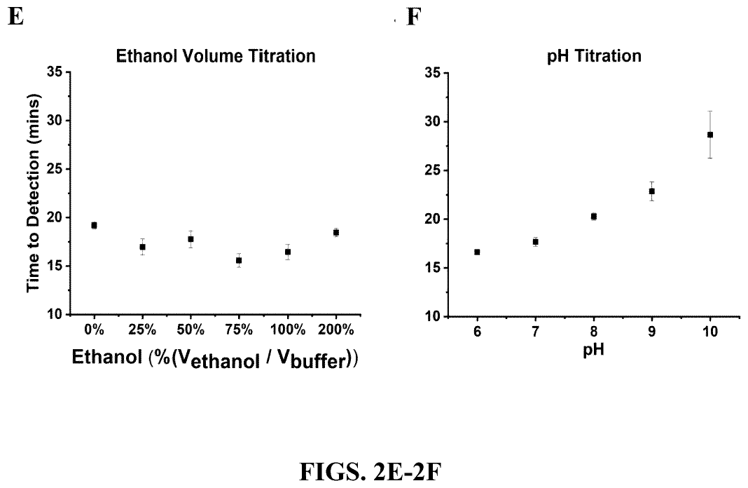

To avoid the need for high salt concentrations and subsequent wash steps to remove excess salt from the silica membrane, we evaluated minimal GuHCl concentrations for binding total nucleic acids without wash steps. We tested a range of GuHCl concentrations to determine the minimal amount required for effective nucleic acid binding to the silica membrane using synthetic SARS-CoV-2 RNA templates (FIG. 2A). Extractions were performed without wash steps to assess the maximum tolerable carryover of GuHCl in the LAMP amplification reaction. The optimal GuHCl concentration was identified as the one yielding the shortest LAMP time-to-detection, indicating minimal inhibition and maximal NA yield. The quickest mean time-to-detection was observed with no GuHCl (0 M) at 17 minutes, and 1 M was the maximum tolerable concentration without inhibiting the LAMP reactions (21.2 minutes). The amplification that happened close to 60 minutes was disregarded due to potential non-specific amplification. Initially, no salt or 1×TE buffer alone gave better amplification without GuHCl, likely due to the EDTA facilitating nucleic acid binding at non-alkaline pH.

In FIG. 2B, we investigated whether the addition of carrier RNA (5.6 μg) could enhance RNA binding to silica. Typically, carrier RNA is used to bind the short or fragmented RNA or is used as a sacrificial RNA to slow down the RNase activity in biofluids.^{49,50} The addition of carrier RNA did not significantly alter the time-to-detection compared to GuHCl alone in the binding buffer; for example, at 1 M GuHCl, with and without carrier RNA resulted in time-to-detection of 20.6 and 21.2 minutes, respectively. There was no significant RNase activity because the templates were spiked in water.

### 2.1.2 Ethanol Volume Titration

In FIG. 2C, we examined the effect of adding ethanol to the binding buffer to enhance nucleic acid binding. Ethanol is commonly used in nucleic acid extraction to remove excess salts and proteins.⁵¹ Additionally, it enhances the interaction between nucleic acids and salts, especially for short RNAs such as micro-RNAs, small interfering RNAs, and fragmented DNA/RNA by reducing the dielectric constant between charged molecules.^{52,53} Moreover, ethanol promotes precipitation by hydrophobic interaction due to its dehydration effect.^{52,53} Therefore, we evaluated the optimal nucleic acid binding by titrating GuHCl concentrations in the presence of ethanol (200 μL). With the addition of ethanol, the fastest mean time-to-detection was observed at 1 M and 2 M GuHCl, at 15.2 and 15.9 minutes, respectively, with minimal variation (p-value=0.14) in detection time while increasing the maximum tolerable concentration up to 4 M GuHCl (21.3 minutes). Subsequently, GuHCl titration was performed with ethanol and carrier RNA combination (FIG. 2D). The results suggest that 2 M GuHCl concentration performed better, amplifying at 13.3 minutes. The p-value between the two lowest times-to-detection, i.e., 1 M and 2 M, is 0.007, suggesting a significant difference. Thus, 2 M GuHCl concentration is significantly better than the rest. It is worth noting that for 2 M GuHCl, by adding ethanol, the final concentration of GuHCl in the sample and buffer matrix reduces to 0.5 M due to the increase in overall volume.

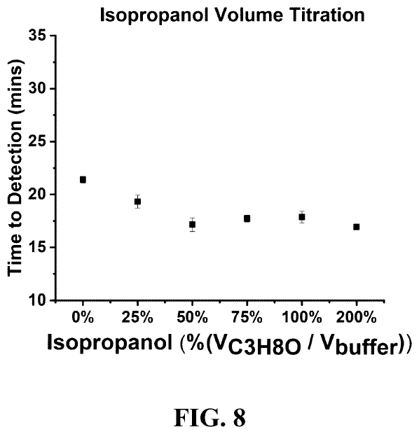

Additionally, we evaluated different volumes of ethanol at 2 M GuHCl concentration to determine whether varying the ethanol-to-sample volume could enhance NA binding. As shown in FIG. 2E, the lowest amplification times were observed at 75% and 100% ethanol volume to sample volume. An ethanol volume equal to the sample volume (100%) was selected for further evaluation due to ease of handling. Similar results were observed with different isopropanol volume ratios (FIG. 8), even though it is assumed that low volume of isopropanol compared to ethanol is needed to precipitate or enhance binding of DNA/RNA.

### 2.1.3 pH Optimization

The pH also plays a crucial role in nucleic acid binding and elution from silica matrix. Typically, low pH enhances NA binding by protonating the silanol group of silica while a high pH deprotonates silica resulting in DNA/RNA elution. 54 Therefore, we evaluated pH ranging from 6 to 10, which is the tolerable buffering range for Tris-EDTA buffers. In FIG. 2F, an increase in pH showed a linear effect (R2=0.94) on nucleic acid binding at 2 M GuHCl concentration. We selected pH 7 for the custom extraction method since pH 6 is outside the 1×TE buffering range. After evaluating different binding conditions, we formulated our custom buffer (CB) with 2 M GuHCl in 1×TE buffer at pH 7. To enhance nucleic acid binding to silica, we added ethanol (1:1 volume to sample) and carrier RNA (5.6 μg) for all subsequent NA extractions.

### 2.2 Viral Lysis and Nucleic Acid Elution Conditions Optimization

### 2.2.1 Lysis Condition

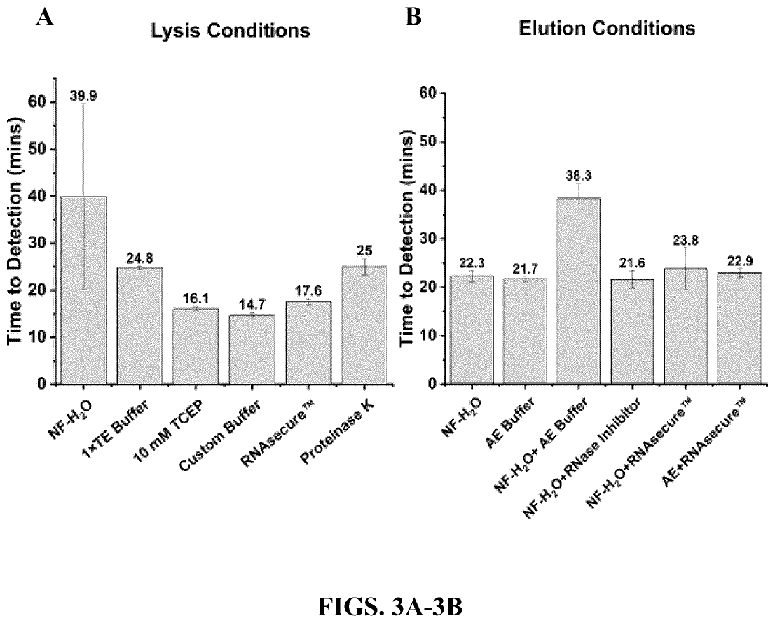

In FIG. 3A, we compared various viral lysis conditions using saliva samples spiked with heat-inactivated SARS-CoV-2 virus particles. The samples were extracted using our custom protocol. Subsequent LAMP analysis demonstrated the efficiency of our custom buffer in viral lysis, yielding faster time-to-detection. Heating the saliva at 95° C. or proteinase K treatment showed high variation between technical replicates in time-to-detection compared to other lysis conditions, likely due to RNA degradation in saliva after viral lysis because of RNases, high temperature, and RNA hydrolysis. The combination of custom buffer (2 M GuHCl in 1×TE) and 10 mM TCEP treatment combined with proteinase K had better RNA stability than heat or proteinase K treatment alone due to its ability to inhibit RNases either by destabilizing disulfide bonds or by chelating RNase cofactors. Thus, the combination of custom buffer with proteinase K performed better than the rest, with a mean time-to-detection of 14.7 minutes.

### 2.2.2 Elution Condition

Furthermore, different elution buffer conditions (FIG. 3B) were evaluated for their impact on LAMP inhibition, RNA retrieval, and RNA degradation, ensuring the recovery of high-quality nucleic acids for accurate diagnostics. EDTA carryover from the extraction process can chelate the ions needed for polymerase activity, and alkaline pH can accelerate RNA degradation through autohydrolysis.^{55,56} Thus, low TE buffer such as AE buffer (Qiagen) was evaluated. Immediate use of eluted samples after extraction showed minimal variation for nuclease-free water and AE as elution buffers. Note that RNA, having a single phosphate backbone, requires only neutral pH (7) to be retrieved, whereas DNA requires higher pH (e.g., 9) such as AE buffer or Low TE buffer for retrieval.

### 2.3 RNase Inactivation in Salivary Samples

Saliva as a biofluid poses many challenges, particularly high RNase activity to protect us from pathogens but is detrimental in nucleic acid extraction and detection. So, we explored simple, fast yet cost-effective enzymatic and chemical nuclease inhibitors. RNases are resistant to heat and chemical denaturation due to the high degree of sulfide bonds stabilizing the tertiary structure of the proteins. Thus, reducing agents such as dithiothreitol (DTT) and Tris(2-carboxyethyl)phosphine (TCEP) are effective choices to disrupt these disulfide bonds in RNase.⁵⁷ While both can reduce disulfide bonds, DTT requires cold storage and is a reversible reducing agent while TCEP is non-reversible and stable at room temperature for a longer duration. Thus, TCEP is a better choice for the RNase inactivation. 58 Additionally, TCEP and DTT can reduce the viscosity of saliva,^{24,59} which can facilitate the handling of biofluids through the silica column in POC devices without the need for high centrifugal force.

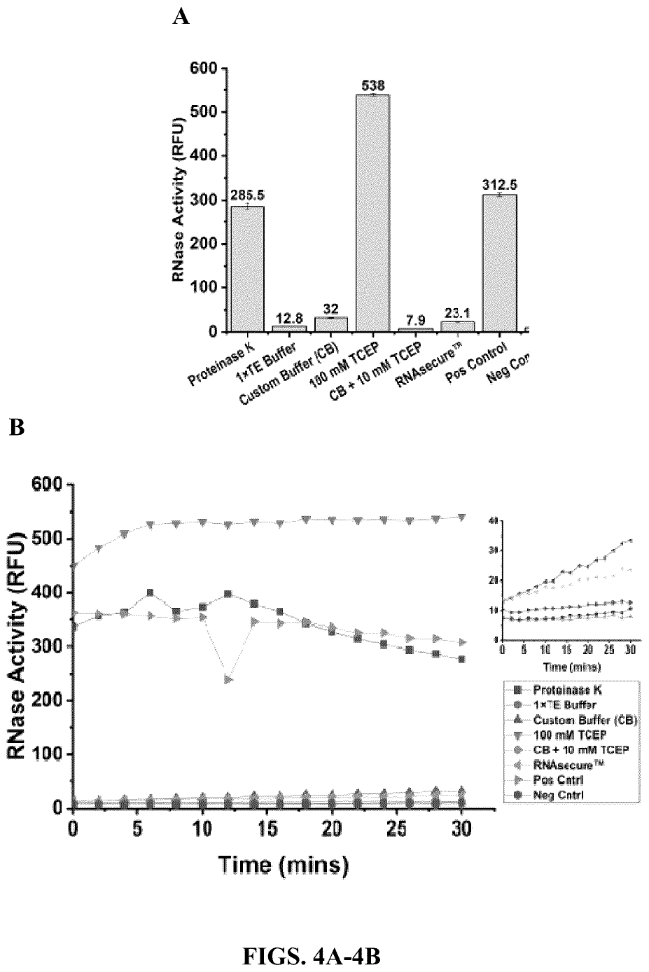

Heat-treated samples were evaluated with different RNase inhibitors to identify the most effective condition using the RNase alert assay (FIG. 4A). Proteinase K treatment alone and 100 mM TCEP showed high mean RFU of 285.5 and 538, respectively, indicating high RNase activity and RNA degradation. The combination of custom buffer with TCEP showed superior performance (7.9 RFU), followed by 1×TE buffer (12.8 RFU). EDTA can chelate metal ions (such as Mg2+, Ca2+, and Mn2+), which are essential cofactors for RNases and other nucleases, thus inhibiting a broad spectrum of nucleases. Murine-based RNase inhibitors are also good alternatives but are costly and require cold storage (data not shown).

FIG. 4B shows the RNase activity measured in terms of relative fluorescence units (RFU) at 37° C. for 30 minutes at 2-minute intervals using RNase Alert assay. 60 The subset (right) shows no significant change in RNase activity between 0 and 30 minutes for CB+10 mM TCEP, similar to the negative control. Therefore, CB with TCEP at 10 mM concentration was selected as an additive for RNase inactivation. Hereafter, the above-optimized custom extraction detailed in methods will be referred to as the POC-Pure (for Point-of-Care Purification) method.

### 2.4 Comparison of Nucleic Acid Extraction Efficiency

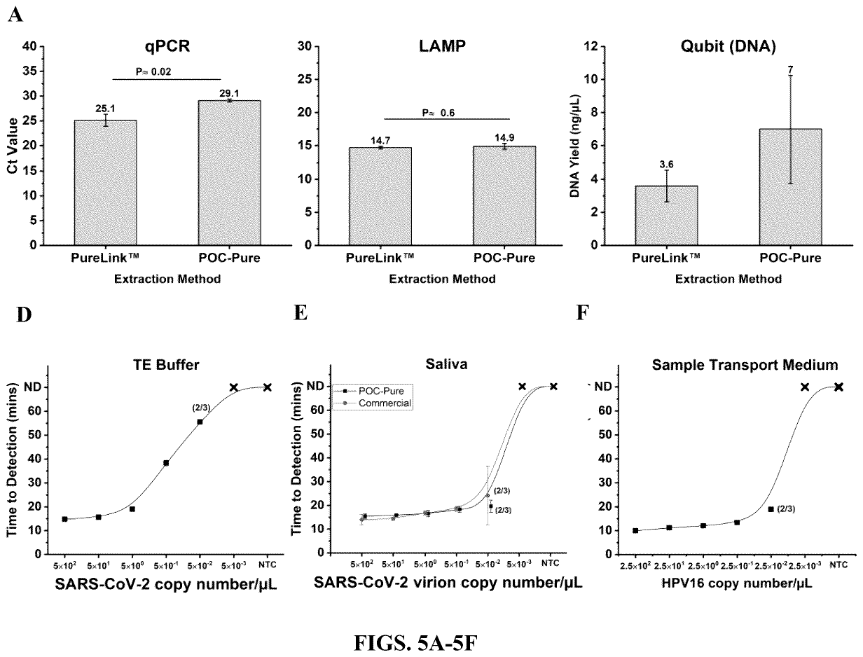

The optimized sample preparation and extraction method, POCPure, was assessed for nucleic acid extraction yield and efficiency using qPCR (Ct values) and LAMP reactions (time-to-detection) by benchmarking it against commercial extraction methods. As shown in FIG. 5A, the POC-Pure method successfully detected RNA but exhibited a slightly higher Ct value compared to the commercial method (p-value=0.02). The extracts were also amplified with LAMP to observe inhibition and extraction efficiency. The FIG. 5B shows that PureLink™ and POC-Pure extraction methods have similar mean times-to-detection of 14.7 and 14.6 minutes, respectively, with no significant difference (p-value=0.6).

Thus, the POC-Pure method demonstrated similar or superior nucleic acid performance to commercial methods without the need for wash steps. This is partly due to the LAMP enhancement effects of carryover GuHCl in the elution. The Qubit™ (ThermoFisher) analysis showed that the POC-Pure method yielded higher DNA amounts (p-value=0.2), mostly due to less shearing and loss of DNA from reduced wash steps and centrifugation. NanoDrop™ (ThermoFisher) analysis showed similar nucleic acid yield between custom and commercial extraction methods (Table 1).

TABLE 1
DNA and RNA yield comparison between PureLink (commercial) extraction
and POC-Pure extraction using 200 μL saliva sample as input

| | DNA yield | | | RNA Yield | | |
|---|---|---|---|---|---|---|
| | (ng/μl) | A260/A280 | A260/A230 | (ng/μl) | A260/A280 | A260/A230 |
| PL 1 | 136.8 | 2.59 | 2.00 | 111.1 | 2.59 | 1.99 |
| PL 2 | 127.7 | 2.62 | 2.08 | 100.1 | 2.62 | 2.06 |
| PL 3 | 154.3 | 2.72 | 2.67 | 79.8 | 2.68 | 2.54 |
| POC-Pure 1 | 117.1 | 2.05 | 0.82 | 103.1 | 1.94 | 0.78 |
| POC-Pure 2 | 114.3 | 2.79 | 1.57 | 88.0 | 2.86 | 1.69 |
| POC-Pure 3 | 127.3 | 2.12 | 0.86 | 91.8 | 2.22 | .92 |

After assessing the efficiency, we evaluated the lower limit of detection (LLOD) of RNA and DNA in 1×TE buffer, saliva, and STM. Whole SARS-CoV-2 viral particles were serially diluted from 100,000 (100K) to 1 copy in 200 μL of sample, and time-to-detection was observed. As shown in FIGS. 5D and 5E, the SARSCoV-2 target was detected in 1×TE buffer and saliva for all three replicates down to 100 copies and 1,000 (1K) copies, respectively. In 1×TE buffer, as low as 10 copies were detected. For the HPV DNA LLOD, 100 copies for all three replicates and as low as 10 copies were detected, as shown in FIG. 5F. Therefore, our custom POC-Pure method has sufficient sensitivity to detect pathogens at significant infectious levels.

### 2.5 On-Chip Extraction Using Customized Rapid Extraction

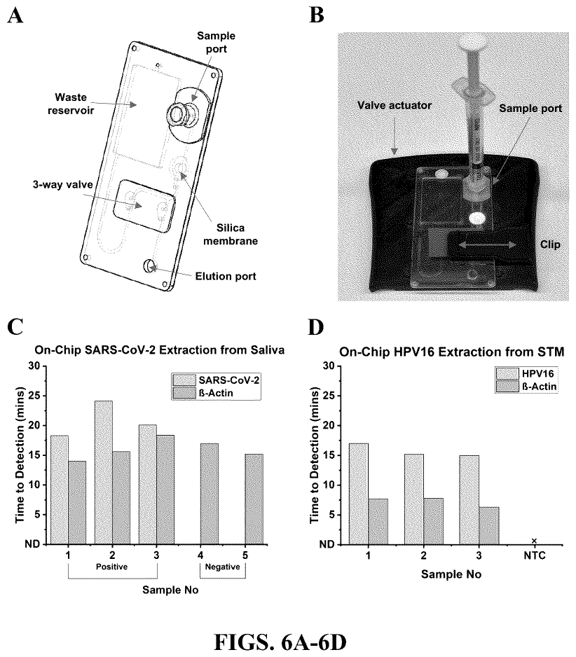

After evaluating the POC-Pure method, we transferred the method to the microfluidic chip shown in FIG. 6A. The chip was designed with a luer lock inlet port, silica matrix, valve system, waste reservoir, and elution port. Syringes containing the sample or buffer are attached to the luer lock so that fluid flow is driven by hand plunging force. FIG. 6B shows a fully assembled microfluidic chip, made with layers of plastic and adhesive films cut using xurographic techniques. The valve actuator platform directs the flow of fluid between the waste reservoir and the elution port.

For on-chip extraction, samples pre-treated with our custom buffer were processed through the microfluidic chip, where nucleic acids were extracted and subsequently analyzed using LAMP assays for specific targets such as SARS-CoV-2 RNA, and HPV 16 DNA. The β-actin (ACTB) was used as an extraction/positive control.

As shown in FIG. 6C, 50,000 copies of SARS-CoV-2 were spiked into three different negative saliva samples and detected with up to a 5-minute variation in time-to-detection. The two negative saliva samples without spiked virus showed no amplification for SARS-CoV-2 RNA targets but successfully amplified for β-actin, indicating that the extraction worked on the microfluidic chip and was specific to the target gene. Additionally, 5,000 SiHa cells were spiked into 200 μL STM and were successfully detected with minimal variation in time-to-detection between three spiked-in samples. The ACTB and HPV 16 detection times were faster for STM since no inhibitors or high RNase activity are present in STM, unlike saliva. No template control (NTC) showed no amplification, suggesting no carryover or cross-contamination.

The custom extraction method developed here could be implemented on other silica-based matrices such as silica-coated paramagnetic beads, glass fiber, and silica gel polymers. This method is also potentially suitable for DNA/RNA extraction from biosamples such as nasal swabs, skin or lesion swabs, urine, plasma, and possibly blood. The microfluidic chip can be modified to split more channels and wells from the silica matrix to detect multiple pathogens or gene targets on a single chip. Table 2 compares the on-chip nucleic acid extraction using the POC-Pure protocol with methods from the literature and commercial methods. As shown in Table 2, on-chip extraction is versatile and can concentrate NA to improve the analytical sensitivity of the integrated sample-to-answer detection.

TABLE 2
Comparison of Nucleic Acid Extraction Methods

| | | | Nucleic Acid | Extraction | |
|---|---|---|---|---|---|
| Method | Sample type | Cell Lysis | Conc. | Time | LOD |
| This work | Saliva, STM, | Chemical and | Yes (Silica) | ~10 mins | 0.25-5 |
| | TE buffer | Thermal | | | copies/μL |
| Cartridge⁶¹ | PBS | Chemical and | Yes (Silica) | ~35 mins | 10³ CFU |
| | | Thermal | | | |
| Saliva | Saliva | Thermal and | No | ~10 mins | 6-12 |
| direct¹⁶ | | Enzymatic | | | copies/μL |
| Oraldisk²⁸ | Saliva | Mechanical and | Yes (Magnetic) | ~40 mins | — |
| | | Chemical | | | |
| Fidget | Urine | — | Yes | ~10 mins | — |
| Spinner²⁷ | | | (Nitrocellulose) | | |
| PureLink⁶² | Saliva, nasal, | Chemical and | Yes (Silica) | ~40 mins | 0.1-10 |
| | buffers | Thermal | | | copies/μL |
| Paper | Blood spot | Chemical | No | ~10 mins | 2.5 × |
| Based²⁶ | | | | | 10³/μL |

### 2.6 Volume and Time Reduction Using Custom Rapid Extraction

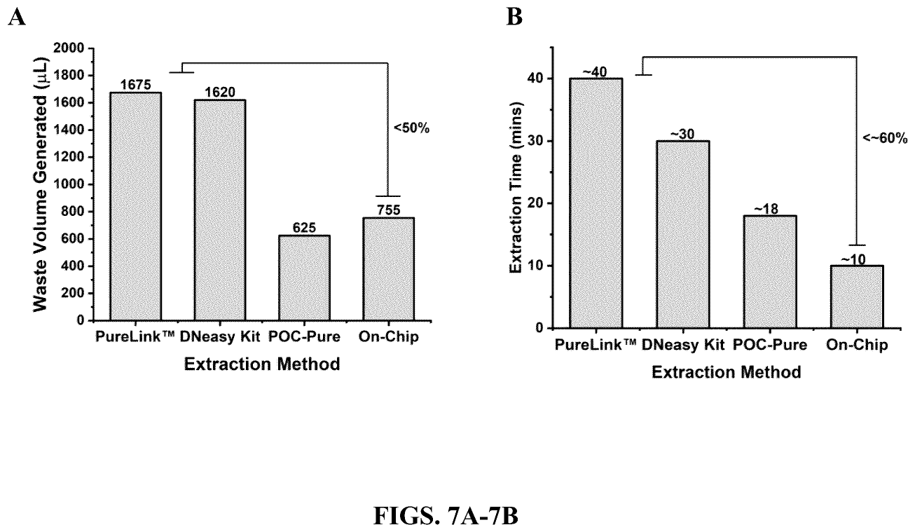

The POC-Pure method results in a significant reduction in the waste volume, as shown in FIG. 7A with more than a 50% decrease in waste volume for a 200 μL sample input. The overall extraction time for a single sample was reduced by about 60% (FIG. 7B), primarily due to less heating time and reduced steps. On-chip extraction was performed using the microfluidic chip in under 10 minutes with a total waste volume of ˜755 μL. Using the traditional extraction method on-chip would lead to 2-3 mL waste volume generation. The elution volume and concentration of the sample cannot be reduced below 50-80 μL with the current design because there is a minimum amount of elution buffer required to wet the entire silica membrane and to retrieve the maximum amount of NA.

### CONCLUSION

The present disclosure describes a robust and fast off-chip and on-chip nucleic acid isothermal and qPCR amplification for SARS-CoV-2 RNA from inactivated virions, and HPV-16 DNA from SiHa cells. The POC Pure extraction method had a detection limit of 100 copies or 0.5 copies/μL of SARS-CoV-2 from 1×TE buffer, 1,000 copies or 5 copies/μL from saliva. For HPV, the detection limits were 50 copies or 0.5 copies/μL of HPV 16 DNA in STM. The POCPure system has been fully migrated to a microfluidic chip. This provides a robust, integrable sample preparation method to address the current challenges of POC molecular testing from biofluids for complete sample-to-answer on portable platforms. Lab-on-chip platforms like this can significantly aid in disease management and public health responses at the point of care, especially in low- and middle-income countries.

### Materials and Methods

### 4.1 Salivary Samples

Saliva samples were collected following protocols approved by the Institutional Review Board (IRB) of Arizona State University (ASU). The ASU Biodesign Clinical Testing Laboratory (ABCTL) used self-collection kits to obtain saliva samples from volunteers throughout the state of Arizona. Collected samples were stored at −80° C. prior to use. All samples were thawed on ice and handled under RNase-free conditions.

### 4.2 Cell Lines and Nucleic Acid Templates

The SiHa (HTB-35™) cell line and heat-inactivated SARS-CoV-2 Omicron variant (VR-3347HK) were procured from the American Type Culture Collection (ATCC, Manassas, VA). SiHa cells were cultured following ATCC protocols using Dulbecco's Modified Eagle Medium (DMEM) supplemented with 10% fetal bovine serum (FBS) and antibiotics. Cells were harvested at 70-80% confluence and preserved in RNAlater (Thermo Fisher Scientific, AM7020) at −80° C. until use.

### Quantitative Genomic RNA of the SARS-CoV-2 Omicron Variant

(VR-3378D) and quantitative synthetic Human papillomavirus 16 (HPV16, VR-3240SD) templates were obtained from ATCC. Viral templates were aliquoted and diluted in nuclease-free water for experimental use.

### 4.3 Loop-Mediated Isothermal Amplification (LAMP)

LAMP primers targeting the orf1a gene of SARS-CoV-2,E6/E7 gene of HPV 16, and β-actin were designed using the New England Biolabs (NEB) primer design tool. Primers were checked for specificity, absence of primer-dimer formation, and amplification efficiency. Synthesized primers (Integrated DNA Technologies, Coralville, IA) were reconstituted in nuclease-free water to 100 μM stock concentrations. A 10× primer mix was prepared by combining 16 μL each of FIP and BIP primers, 2 μL each of F3 and B3 primers, and 4 μL each of LF and/or LB primers, bringing the total volume to 100 μL with nuclease-free water.

LAMP reactions were set up in a dedicated PCR hood to prevent cross-contamination. Each 20 μL reaction contained 10 μL of 2×Warm Start LAMP Master Mix (NEB, E1700), 2 μL of 10× primer mix, 0.4 μL of SYTO-9 fluorescent dye (250 μM, Life Technologies, S34854), 0.1 μL of dUTP (NEB, NO459S), 0.4 μL of Antarctic Thermolabile UDG (NEB, M0372S), and 5 μL of template or nuclease-free water (negative control). β-actin (ACTB) was used as a positive control.

Reactions were incubated at 65° C. for 60 minutes on a ViiA 7 Real-Time PCR System (Applied Biosystems) or an open qPCR machine (CHAI, Santa Clara, CA), with real-time fluorescence data collected every 30 seconds. Post-amplification, tubes were not opened to prevent contamination.

4.4 Quantitative PCR (qPCR)

SARS-CoV-2-specific primer-probe mix (VIC dye) and RNase P (JUN dye) as a positive control were acquired from Thermo Fisher Scientific (Applied Biosystems, A51606). HPV 16-specific primers and probes were synthesized based on previously published sequences (63) (Integrated DNA Technologies). Each 20 μL qPCR reaction comprised 10 μL of 2× TaqMan Fast Advanced Master Mix (Applied Biosystems, 4445566), 1 μL of 20× primer-probe mix (900 nM primers, 250 nM probe in 1× TE buffer), and 9 μL of template or nuclease-free water. Thermal cycling conditions were 50° C. for 2 minutes, 85° C. for 10 minutes, followed by 40 cycles of 95° C. for 15 seconds and 60° C. for 1 minute. Reactions were performed on a ViiA 7 Real-Time PCR System (Applied Biosystems).

### 4.5 Development of Custom Extraction Protocol

### 4.5.1 Custom Buffer Preparation

A custom buffer (CB) containing 2M Guanidine HCl, 10 mM Tris-HCl, and 1 mM EDTA was prepared by dissolving 19.106 g of Guanidine HCl (Millipore Sigma, G3272, St. Louis, MO), 0.1576 g of Tris-HCl (Millipore Sigma, 10812846001, St. Louis, MO), and 0.0372 g of EDTA (Millipore Sigma, E7889, St. Louis, MO) in 70 mL of Milli-Q® water. The volume was adjusted to 100 mL, and the solution was heated at 65° C. for 5 minutes to dissolve Guanidine HCl. After cooling, the pH was adjusted to 7.0 using 1N HCl.

### 4.5.2 Determination of Optimal GuHCl Concentration

To determine the effective GuHCl concentration for nucleic acid binding without wash steps, solutions of GuHCl at concentrations of 0 M, 1 M, 2 M, 4 M, 8 M, and 10M were prepared in 1×TE buffer (10 mM Tris-HCl, 1 mM EDTA, pH 7.0). Synthetic SARSCoV-2 RNA (50,000 copies) was spiked into 200 μL of nuclease-free water and mixed with 200 μL of each GuHCl solution.

The mixtures were loaded into Zymo IIC-XLR silica columns (Zymo Research, C1104-50) and centrifuged at 8,000×g for 1 minute at room temperature. After discarding the flow-through, 50 μL of nuclease-free water was added to elute the RNA at 13,300×g. Extracts (5 μL) were used in LAMP reactions to measure time-to-detection across different GuHCl concentrations.

### 4.5.3 Evaluation of Carrier RNA and Ethanol

To assess the effect of carrier RNA, 5.6 μg of carrier RNA (Applied Biosystems, 4382878, Lithuania) was added to each GuHCl solution, and the extraction and detection procedures were repeated.

The role of ethanol was explored by adding 200 μL of ethanol (200 proof) to the GuHCl solutions, followed by extraction and LAMP analysis. Combinations of ethanol and carrier RNA were also tested to identify optimal conditions that avoid inhibition.

A titration series of ethanol volumes (0%, 25%, 50%, 75%, and 100% of sample volume) was evaluated at 2M GuHCl concentration to optimize nucleic acid binding.

### 4.5.4 pH Optimization

The pH of 2M GuHCl solutions in 1×TE buffer was adjusted to pH 6, 7, 8, 9, and 10 using 1N HCl or 10N NaOH to determine the optimal pH for nucleic acid binding. Hereafter, for all subsequent NA extractions, we used our custom buffer (CB) with 2M GuHCl in 1×TE buffer at pH 7.

### 4.6 Viral Lysis, Elution Efficiency, and RNase Inactivation

### 4.6.1 Preparation of Reagents

To prepare the TCEP solution (100 mM TCEP+50 mM EDTA), 0.28 g of Tris(2-carboxyethyl)phosphine hydrochloride (TCEPHCI; Millipore Sigma, C4706, St. Louis, MO, USA) and 0.14 g of EDTA (Millipore Sigma, E7889, St. Louis, MO, USA) were dissolved in 5 mL of Milli-Q® water. The volume was then adjusted to 10 mL with Milli-Q® water, and the pH was brought to 7.0 using 1N HCl.

For the RNAsecure™ solution used in lysis evaluation, 800 μL of 25×RNAsecure™ reagent (Invitrogen, AM7005, Lithuania) was mixed with 9.2 mL of nuclease-free water to make a total volume of 10 mL. The proteinase K solution was prepared by adding 250 μL of proteinase K (Qiagen, 19131, Hilden, Germany) to 750 μL of nuclease-free water.

### 4.6.2 Viral Lysis and Extraction

In the viral lysis experiments, 200 μL of pooled negative saliva samples spiked with 50,000 copies of heat-inactivated SARS-CoV-2 (Omicron variant) virus was combined with 200 μL of one of the following solutions: nuclease-free water; proteinase K solution prepared as 25 μL proteinase K mixed with 175 μL nuclease-free water; 1×TE buffer (Invitrogen, AM9858, Lithuania); a 10 mM TCEP solution made by mixing 20 μL of the TCEP solution from section 4.6.1, 25 μL proteinase K, and 155 μL of 1×TE buffer; the custom buffer (CB); or the RNAsecure™ solution prepared earlier. Subsequently, 5.6 μL of carrier RNA (Applied Biosystems, 4382878, USA) was added to each mixture. Then the samples were thoroughly mixed by vortexing for 30 seconds and then heated at 95° C. for 5 minutes to lyse viral particles and inactivate enzymes. After cooling to room temperature, the samples were extracted using the steps in section 4.5.2 as described previously and analyzed as outlined in section 4.3.

### 4.6.3 Elution Efficiency Evaluation

To evaluate elution efficiency, 200 μL of the custom buffer was mixed with 200 μL of the pooled negative saliva sample spiked with 50,000 copies of heat-inactivated SARS-CoV-2 (Omicron variant) virus. Following the standard extraction procedure, nucleic acids were eluted using 50 μL of different elution buffers. The buffers used were nuclease-free water; AE buffer (Qiagen, 19077, Hilden, Germany); a mixture of 45 μL nuclease-free water with 5 μL Low TE buffer; a mixture of 45 μL nuclease-free water with 5 μL RNase Inhibitor (Applied Biosystems, N8080119, Lithuania); a mixture of 45 μL nuclease-free water with 5 μL RNAsecure™; and a mixture of 45 μL Low TE buffer with 5 μL RNAsecure™. The extracted nucleic acids were immediately used in LAMP reactions as described in section 4.3 to assess the impact of different elution buffers on amplification efficiency and RNA integrity.

### 4.6.4 RNase Inactivation Assay

To determine the optimal RNase inactivation conditions, 200 μL of a pooled negative saliva sample was combined with 200 μL of various solutions, each designed to inactivate RNases effectively. The solutions included proteinase K solution as prepared in section 4.6.1; 1×TE buffer; the custom buffer (CB); 100 mM TCEP solution from section 4.6.1; custom buffer mixed with 10 mM TCEP and 5 mM EDTA; and an RNAsecure™ solution made by mixing 180 μL of nuclease-free water with 20 μL of RNAsecure™. The mixtures were vortexed and then heat-treated at 95° C. for 5 minutes to denature proteins and inactivate RNases. Samples were subsequently cooled on ice for 2 minutes.

After cooling, 45 μL of each heat-treated sample was mixed with 5 μL of 10×RNase Alert substrate (Invitrogen, AM1964, Lithuania). For the positive control, 5 μL of RNase control from the kit was combined with 40 μL of nuclease-free water and 5 μL of RNase Alert substrate. The negative control was prepared by mixing 45 μL of nuclease-free water with 5 μL of RNase Alert substrate. All samples and controls were incubated at 37° C. for 30 minutes.

Relative fluorescence units (RFU), indicating RNase activity, were measured using a SpectraMax® M5 spectrophotometer (Molecular Devices, Sunnyvale, CA, USA) with excitation and emission wavelengths set at 490 nm and 520 nm, respectively, and the photomultiplier gain set to low. Additionally, to monitor RNase activity over time, RFU readings were collected every 2 minutes throughout the 30-minute incubation period.

### 4.7 Custom Extraction Method (POC-Pure)

The optimized custom extraction method (POC-Pure) involved adding CB with 10 mM TCEP and 5 mM EDTA to the salivary sample in a 1:1 volume ratio (e.g., 200 μL sample+200 μL CB with 10 mM TCEP and 5 mM EDTA). The mixture was vortexed for 30 seconds and heated at 95° C. for 5 minutes to lyse cells and inactivate RNases. After cooling to room temperature, ethanol was added in a 1:1 ratio to the sample volume (200 μL). The sample was loaded into a silica column and centrifuged at 8,000×g for 1 minute. Nucleic acids were eluted with 50 μL of nuclease-free water (for RNA) or AE buffer (for DNA) by centrifuging at 13,300×g or maximum speed.

### 4.8 Nucleic Acid Extraction Efficiency Comparison

To compare the POC-Pure method with a commercial kit, 200 μL of pooled negative saliva spiked with 50,000 copies of heat-inactivated SARS-CoV-2 was extracted using both the POC-Pure method and the PureLink™ Viral RNA/DNA Mini Kit (Invitrogen, 12280-050). Nucleic acids were eluted in 50 μL of nuclease-free water. Extracts (5 μL) were analyzed using qPCR and LAMP assays as described above.

DNA yield was quantified using the Qubit™ dsDNA HS Assay Kit (Invitrogen, Q33231) on a Qubit™ Flex fluorometer (Invitrogen, Q33327). Nanodrop™ (ThermoFisher, ND-ONE-W) spectrophotometry was used to assess purity ratios (A260/280 and A260/230).

### 4.9 Limit of Detection Determination

For SARS-CoV-2, synthetic RNA and heat-inactivated virus were serially diluted to concentrations of 0, 1, 10, 100, 1,000, 10,000, and 100,000 copies in 200 μL of 1×TE buffer and pooled negative saliva, respectively. Extractions were performed using the POC-Pure method, and LAMP assays were conducted to determine the lower limit of detection (LLOD).

For HPV16 DNA, synthetic HPV16 DNA templates were serially diluted to concentrations of 0, 1, 10, 100, 1,000, and 10,000 copies in 200 μL of specimen transport medium (STM). Extractions were performed using AE buffer, and LAMP assays were conducted to determine the LLOD. Each concentration was tested in triplicate to assess reproducibility and consistency.

### 4.10 Microfluidic Chip Fabrication

The microfluidic chip was fabricated using laser-cutting techniques. The middle layer consisted of 1.5 mm clear cast acrylic (McMaster-Carr) laser-cut with features for the inlet port, silica matrix (Zymo-Spin III column), valves, waste reservoir, and elution chamber. Adhesive films (Adhesives Research, 90445Q) were cut using a Cricut Explore cutting plotter to create fluidic channels leading to each feature.

The adhesive layers were laminated on both sides of the acrylic, embedding the silica matrix between them. Polyester film (Melinex 454 PET, Tekra) was laminated on the outer surfaces to seal the channels and features. Silicone rubber (McMaster-Carr, 1460N11) was cut to cover the valves, and a luer lock attachment was 3D-printed (Formlabs Form 3) using clear resin and affixed over the inlet port.

### 4.11 On-Chip Extraction Procedure

For on-chip nucleic acid extraction, 200 μL samples of negative saliva spiked with 50,000 copies of heat-inactivated SARS-CoV-2 or STM spiked with 5,000 SiHa cells were mixed with an equal volume of custom buffer (with 10 mM TCEP added only for saliva samples) and heated at 95° C. for 5 minutes. After cooling, samples were loaded into 3 mL luer-lock syringes (Becton Dickinson, 309657) and connected to the chip's inlet port.

The valve was positioned to direct flow toward the waste reservoir. The sample was manually injected through the silica matrix into the waste reservoir. Subsequently, a 1 mL syringe (JD+01L, Nipro) containing 140 μL of nuclease-free water (for saliva samples) or 200 μL of AE buffer (for STM samples) was used to flush residual sample from the silica matrix into the waste.

For elution, a syringe containing 220 μL of nuclease-free water (RNA) or AE buffer (DNA) was connected, and the valve was adjusted to direct flow toward the elution port. Eluate was collected in 20 μL fractions up to a total volume of 100 μL. Extracts (5 μL) were used in LAMP assays targeting SARS-CoV-2, HPV 16, and β-actin.

### 4.12 Statistical Analysis

All experiments were performed in triplicate unless otherwise stated. Data are presented as mean±} standard deviation (SD). Comparisons between two groups were performed using un-paired two-tailed Student's 1-tests to determine statistical significance. Linear regression analysis was employed using Origin software to evaluate the relationship between pH levels and nucleic acid binding efficiency, with the correlation coefficient (R2) indicating the strength of the association. Time-to-detection in LAMP assays and cycle threshold (Ct) values in qPCR assays were analyzed to compare the performance of different extraction methods and conditions. Statistical significance was determined by comparing the mean values obtained under different conditions. A p-value less than 0.05 was considered statistically significant.

### Abbreviations

AE: Aqueous Elution, Qiagen
CB: Custom Buffer
COVID-19: Coronavirus Disease 2019
DNA: Deoxyribonucleic Acid
DTT: Dithiothreitol
EDTA: Ethylenediaminetetraacetic Acid
GuHCl: Guanidine Hydrochloride
HDA: Helicase Dependent Amplification
HPV: Human Papillomavirus
LAMP: Loop-Mediated Isothermal Amplification
LOD: Limit of Detection
NA: Nucleic Acid.
PCR: Polymerase Chain Reaction.
POC: Point-of-Care
qPCR: Quantitative Polymerase Chain Reaction
RNA: Ribonucleic Acid
RPA: Recombinase Polymerase Amplification
RNase: Ribonuclease
SARS-CoV-2: Severe acute respiratory syndrome coronavirus 2
STM: Specimen Transport Medium
TCEP: Tris(2-carboxyethyl)phosphine
TE: Tris-EDTA

### REFERENCES

1. S. C. Tan and B. C. Yiap, BioMed Research International, 2009, 2009, 574398.
2. M. N. Emaus, M. Varona, D. R. Eitzmann, S.-A. Hsieh, V. R. Zeger and J. L. Anderson, TrAC Trends in Analytical Chemistry, 2020, 130, 115985.
3. M. L. Everitt, A. Tillery, M. G. David, N. Singh, A. Borison and I. M. White, Analytica Chimica Acta, 2021, 1146, 184-199.
4. A. Hosseini, R. Pandey, E. Osman, A. Victorious, F. Li, T. Didar and L. Soleymani, ACS sensors, 2020, 5, 3328-3345.
5. D. R. Peaper, C. A. Kerantzas and T. J. Durant, Clinical Bio-chemistry, 2023, 117, 94-101.
6. J. Y. Zhang, A. T. Bender, D. S. Boyle, P. K. Drain and J. D. Posner, Analyst, 2021, 146, 2449-2462.
7. N. Ali, R. d. C. P. Rampazzo, A. D. T. Costa and M. A. Krieger, BioMed research international, 2017, 2017, 9306564.
8. R. Paul, E. Ostermann and Q. Wei, Biosensors and bioelectron-ics, 2020, 169, 112592.
9. M. Zamani, A. L. Furst and C. M. Klapperich, Accounts of chem-ical research, 2021, 54, 3772-3779.
10. K. To, C. Yip, C. Lai, C. Wong, D. Ho, P. Pang, A. Ng, K.-H. Leung, R. Poon, K.-H. Chan et al., Clinical Microbiology and Infection, 2019, 25, 372-378.
11. C. S. Laxton, C. Peno, A. M. Hahn, O. M. Allicock, S. Perniciaro and A. L. Wyllie, The Lancet Microbe, 2023, 4, e837-e850.
12. M. Goh, C. Joy, A. N. Gillespie, Q. R. Soh, F. He and V. Sung, Pediatric Research, 2024, 95, 508-531.
13. A. A. Chen, T. Gheit, M. Stellin, V. Lupato, G. Spinato, R. Fu-son, A. Menegaldo, S. Mckay-Chopin, E. Dal Cin, G. Tirelli et al., Oral Oncology, 2017, 75, 106-110.
14. T. W. Pittman, D. B. Decsi, C. Punyadeera and C. S. Henry, Theranostics, 2023, 13, 1091.
15. Y. Cui, H.-S. Kim, E. S. Cho, D. Han, J. A. Park, J. Y. Park, W. Nam, H. J. Kim, I.-H. Cha and Y. H. Cha, PloS one, 2021, 16, e0256979.
16. C. B. Vogels, A. E. Watkins, C. A. Harden, D. E. Brackney, J. Shafer, J. Wang, C. Caraballo, C. C. Kalinich, I. M. Ott, J. R. Fauver et al., Med, 2021, 2, 263-280.
17. C.-C. Tseng, C.-T. Kung, R.-F. Chen, M.-H. Tsai, H.-R. Chao, Y.-N. Wang and L.-M. Fu, Sensors and Actuators B: Chemical, 2021, 342, 130078.
18. F. Ferrara, S. Zoupanou, E. Primiceri, Z. Ali and M. S. Chiri-acò, Biosensors and Bioelectronics, 2022, 196, 113698.
19. N. Garg, D. Boyle, A. Randall, A. Teng, J. Pablo, X. Liang, D. Camerini and A. P. Lee, Lab on a Chip, 2019, 19, 1524-1533.
20. A. Wozniak, A. Cerda, C. Ibarra-Henriquez, V. Sebastian, G. Armijo, L. Lamig, C. Miranda, M. Lagos, S. Solari, A. M. Guzmán et al., Scientific Reports, 2020, 10, 16608.
21. V. Genoud, M. Stortz, A. Waisman, B. G. Berardino, P. Verneri, V. Dansey, M. Salvatori, F. Remes Lenicov and V. Levi, PLOS One, 2021, 16, e0247792.
22. J. Qian, S. A. Boswell, C. Chidley, Z.-x. Lu, M. E. Pettit, B. L. Gaudio, J. M. Fajnzylber, R. T. Ingram, R. H. Ward, J. Z. Li et al., Nature Communications, 2020, 11, 5920.
23. D. R. Solovchuk, I. H. Boyaci, U. Tamer, N. Sahiner and D. çetin, Microchemical Journal, 2023, 189, 108479.
24. B. A. Rabe and C. Cepko, Proceedings of the National Academy of Sciences, 2020, 117, 24450-24458.
25. M. G. Mason and J. R. Botella, Nature Protocols, 2020, 15, 3663-3677.
26. K. Malpartida-Cardenas, J. Baum, A. Cunnington, P. Georgiou and J. Rodriguez-Manzano, Analyst, 2023, 148, 3036-3044.
27. I. Michael, D. Kim, O. Gulenko, S. Kumar, S. Kumar, J. Clara, D. Y. Ki, J. Park, H. Y. Jeong, T. S. Kim et al., Nature biomedical engineering, 2020, 4, 591-600.
28. D. Baumgartner, B. Johannsen, M. Specht, J. Luddecke, M. Rombach, S. Hin, N. Paust, F. von Stetten, R. Zengerle, C. Herz et al., Biosensors, 2021, 11, 423.
29. S. Mondal, N. Feirer, M. Brockman, M. A. Preston, S. J. Teter, D. Ma, S. A. Goueli, S. Moorji, B. Saul and J. J. Cali, Science of the Total Environment, 2021, 795, 148834.
30. B. S. Batule, Y. Seok and M.-G. Kim, Biosensors and Bioelec-tronics, 2020, 151, 111998.
31. A. Neuville, L. Renaud, T. T. Luu, M. W. Minde, E. Jettestuen, J. L. Vinningland, A. Hiorth and D. K. Dysthe, Lab on a Chip, 2017, 17, 293-303.
32. M. Mahalanabis, J. Do, H. ALMuayad, J. Y. Zhang and C. M. Klapperich, Biomedical microdevices, 2010, 12, 353-359.
33. D. A. Bartholomeusz, R. W. Boutté and J. D. Andrade, Journal of Microelectromechanical systems, 2005, 14, 1364-1374.
34. S. Shijo, D. Tanaka, T. Sekiguchi, J.-i. Ishihara, H. Takahashi, M. Kobayashi and S. Shoji, Micromachines, 2023, 14, 706.
35. M. L. Cunha, S. S. da Silva, M. C. Stracke, D. L. Zanette, M. N. Aoki and L. Blanes, Analytical Chemistry, 2021, 94, 41-58.
36. Z. Li, X. Xu, D. Wang and X. Jiang, TrAC Trends in Analytical Chemistry, 2023, 158, 116871.
37. D. Obino, M. Vassalli, A. Franceschi, A. Alessandrini, P. Facci and F. Viti, Sensors, 2021, 21, 3058.
38. M. M. Islam and D. Koirala, Analytica chimica acta, 2022, 1209, 339338.
39. P. Rodriguez-Mateos, B. Ngamsom, C. Walter, C. E. Dyer, J. Gi-taka, A. Iles and N. Pamme, Analytica chimica acta, 2021, 1177, 338758.
40. D. Das, C.-W. Lin and H.-S. Chuang, Biosensors, 2022, 12, 1068.
41. W. Witkowska McConnell, C. Davis, S. R. Sabir, A. Garrett, A. Bradley-Stewart, P. Jajesniak, J. Reboud, G. Xu, Z. Yang, R. Gunson et al., Nature communications, 2021, 12, 6994.
42. G. Papadakis, A. K. Pantazis, N. Fikas, S. Chatziioannidou, V. Tsiakalou, K. Michaelidou, V. Pogka, M. Megariti, M. Var-daki, K. Giarentis et al., Scientific reports, 2022, 12, 3775.
43. S. M. Lee, H. K. Balakrishnan, E. H. Doeven, D. Yuan and R. M. Guijt, Biosensors, 2023, 13, 980.
44. T. Poeckh, S. Lopez, A. O. Fuller, M. J. Solomon and R. G. Larson, Analytical biochemistry, 2008, 373, 253-262.
45. P. Chomczynski and N. Sacchi, Nature protocols, 2006, 1, 581-585.
46. J. M. Chirgwin, A. E. Przybyla, R. J. MacDonald and W. J. Rutter, biochemistry, 1979, 18, 5294-5299.
47. Y. Zhang, G. Ren, J. Buss, A. J. Barry, G. C. Patton and N. A. Tanner, Biotechniques, 2020, 69, 178-185.
48. G. Alhamid, H. Tombuloglu and E. Al-Suhaimi, Scientific Re-ports, 2023, 13, 5066.
49. K. J. Shaw, L. Thain, P. T. Docker, C. E. Dyer, J. Greenman, G. M. Greenway and S. J. Haswell, Analytica chimica acta, 2009, 652, 231-233.
50. D. Andreasen, J. U. Fog, W. Biggs, J. Salomon, I. K. Dahslveen, A. Baker and P. Mouritzen, Methods, 2010, 50, S6-S9.
51. N. Sun, C. Deng, Y. Liu, X. Zhao, Y. Tang, R. Liu, Q. Xia, W. Yan and G. Ge, Journal of chromatography A, 2014, 1325, 31-39.
52. S. He, B. Cao, Y. Yi, S. Huang, X. Chen, S. Luo, X. Mou, T. Guo, Y. Wang, Y. Wang et al., Nano Select, 2022, 3, 617-626.
53. Y. Li, S. Chen, N. Liu, L. Ma, T. Wang, R. N. Veedu, T. Li, F. Zhang, H. Zhou, X. Cheng et al., BioTechniques, 2020, 68, 191-199.
54. M. A. Brown, F. Vila, M. Sterrer, S. Thuurmer, B. Winter, M. Ammann, J. J. Rehr and J. A. van Bokhoven, The journal of physical chemistry letters, 2012, 3, 1754-1759.
55. R. Bock, Methods in enzymology, 1967, 12, 224-228.
56. A. Kuffel, A. Gray and N. N. Daeid, International journal of legal medicine, 2021, 135, 63-72.
57. I. B. Santarino, S. C. B. Oliveira and A. M. Oliveira-Brett, Elec-trochemistry Communications, 2012, 23, 114-117.
58. J. C. Han and G. Y. Han, Analytical biochemistry, 1994, 220, 5-10.
59. A. J. Fischer, M. I. Pino-Argumedo, B. M. Hilkin, C. R. Shan-rock, N. D. Gansemer, A. L. Chaly, K. Zarei, P. D. Allen, L. S. Ostedgaard, E. A. Hoffman et al., JCI insight, 2019, 4, e124863.
60. A. T. Bender, B. P. Sullivan, L. Lillis and J. D. Posner, The Journal of Molecular Diagnostics, 2020, 22, 1030-1040.
61. W. Han, J. Shin, J. H. Shin et al., Sensors and Actuators B: Chemical, 2023, 375, 132948.
62. A. Bordigoni, A. Motte, H. Tissot-Dupont, P. Colson and C. Desnues, Scientific Reports, 2021, 11, 4039.
63. S. Jeong, E. González-Grandío, N. Navarro, R. L. Pinals, F. Ledesma, D. Yang and M. P. Landry, ACS nano, 2021, 15, 10309-10317.

1. A composition for extracting nucleic acids from a sample, the composition comprising:
a chaotropic agent at a concentration of between 1M and about 4M; and
a Tris-EDTA buffer comprising about 10 mM Tris-HCl and between about 0.1 and about 10 mM EDTA.

2. The composition of claim 1, wherein the chaotropic agent is guanidine hydrochloride (GuHCl); and wherein the GuHCl is at a concentration of between about 1 M and about 2 M.

3. The composition of claim 1, wherein the chaotropic agent is sodium chloride (NaCl) or lithium chloride (LiCl).

4. The composition of claim 1, wherein the EDTA is at a concentration of about 1 mM.

5. The composition of claim 1, further comprising tris(2-carboxyethyl)phosphine TCEP at a concentration of between about 1 mM and about 100 mM.

6. The composition of claim 5, wherein the TCEP is at a concentration of about 10 mM.

7. The composition of claim 1, wherein a pH of the composition is between about 6 and about 8.

8. The composition of claim 7, wherein the pH is about 7.

9. The composition of claim 1, further comprising at least one of an RNase inhibitor and a proteinase.

10. A kit comprising:
the composition of claim 1;
a carrier RNA; and
a precipitating agent.

11. The kit of claim 10, wherein the precipitating agent is ethanol or polyethylene-glycol (PEG).

12. A method for extracting nucleic acids from a biological sample, the method comprising:
a) combining equal volumes of the sample with the composition of claim 1 to create a mixture;
b) incubating the mixture at between about 55° C. and about 95° C. for between about 30 seconds to about 10 minutes;
c) cooling the mixture to room temperature; and
d) separating the nucleic acids from the mixture.

13. The method of claim 12, further comprising adding a carrier RNA to the mixture before step b).

14. The method of claim 12, further comprising adding PEG at a concentration of about 10% w/v to the mixture before step b).

15. The method of claim 12, further comprising adding ethanol or isopropanol to the mixture at a ratio of about 1:1 after step c) and before step d); wherein the ethanol is about 100% ethanol; and wherein the isopropanol is about 25% to about 100% isopropanol.

16. The method of claim 12, wherein step b) is performed at about 95° C. for about 5 minutes.

17. The method of claim 12, wherein step d) comprises adding the mixture to an extraction agent to separate the nucleic acids from the remaining mixture; discarding the remaining mixture; adding an elution buffer to the extraction agent to create an eluate comprising the nucleic acids; and collecting the eluate.

18. The method of claim 17, wherein the extraction agent is selected from paramagnetic silica beads, glass fiber sheets, and glass silica beads.

19. The method of claim 12, further comprising analyzing the nucleic acids using at least one of loop-mediated amplification (LAMP), recombinase polymerase amplification (RPA), quantitative PCR, and nucleic acid sequencing.

20. The method of claim 12, wherein the biological sample comprises saliva.
